(19) United States
(12) Patent Application Publication
(10) Pub. No.: US 2009/0222032 A1
(43) Pub. Date: Sep. 3, 2009

(54) DEVICE AND METHOD OF WEIGHT CONTROL VIA INDIRECT ABDOMINAL CAVITY VOLUME REDUCTION

(76) Inventor: Gary M. Horndeski, League City, TX (US)

Correspondence Address: FISH & RICHARDSON P.C., P.O. BOX 1022, MINNEAPOLIS, MN, 55440-1022, US

(21) Appl. No.: 12/466,928

(22) Filed: May 15, 2009

Related U.S. Application Data
(63) Continuation-in-part of application No. 11/355,126, filed on Feb. 15, 2006.
Application No. 11/355,126 is a continuation-in-part of application No. 11/091,127, filed on Mar. 28, 2005.
Application No. 11/091,127 is a continuation-in-part of application No. 11/091,127, filed on Mar. 28, 2005.

(51) Int. Cl. A61B 17/08

(52) U.S. Cl. 606/157

(57) ABSTRACT

A device for controlling the weight of a body can be a passive or expandable food restriction device. The presence of the device decreases abdominal wall compliance and increases intra-abdominal pressure thereby producing satiety. One embodiment of the expandable device includes a hollow member with an expandable and a non-expandable portion to its exterior surface. The hollow member is selectively expanded and/or contracted following implantation in order to maintain pressure in the abdominal cavity. Additionally, a surgical method applying two or more versions of the food restriction device is described, such that multiple passive and/or expandable devices may be inserted into the body for additional weight control measures. The device is preferably positioned superficial to the abdominal cavity according to the present disclosure to minimize the risks associated with traditional food intake restriction surgeries.

### CROSS-REFERENCE TO RELATED APPLICATION

This application is a continuation-in-part of, and therefore claims priority to, U.S. patent application Ser. No. 11/355,126 filed on Feb. 15, 2006, which is a continuation-in-part and claims priority to U.S. patent application Ser. No. 11/091,127, filed on Mar. 28, 2005, which are incorporated herein by reference for all purposes. This Application is also a continuation-in-part and therefore claims priority to U.S. patent application Ser. No. 11/091,127, filed on Mar. 28, 2005, all of which is incorporated herein by reference for all purposes.

### TECHNICAL FIELD

This disclosure relates to weight control, and more particularly to a device and method for controlling body weight via indirect reduction of the volume of the abdominal cavity.

### BACKGROUND

Obesity is a chronic disease and constitutes a major health concern. In the United States alone obesity accounts for more than $100 billion in health care annually. Far from being a purely cosmetic issue, being obese or morbidly obese puts a person at an increased risk for developing and/or aggravating dozens of serious medical conditions. More than 30 obesity-related medical conditions are currently recognized. These include arthritis, several forms of cancer, carpal tunnel syndrome, cardiovascular disease, gallbladder disease, gout, hypertension, infertility, liver disease, low back pain, obstetric and gynecologic complications, sleep apnea, stroke, type-2 diabetes, and urinary stress incontinence.

Obesity is commonly measured by using the Body Mass Index (BMI). In terms of BMI, obesity is defined as having a BMI of 30 kg/m². Morbid obesity is defined as the condition of obesity coupled with one or more secondary debilitating factors, such as hypertension, cardiovascular disease, and/or diabetes. A BMI of 40 kg/m² is generally recognized to constitute morbid obesity. Importantly, morbid obesity ranks second only to smoking as a preventable cause of death in the U.S.

While obesity is recognized to be simply an imbalance between caloric intake and caloric expenditure, the factors producing obesity are varied and complex. Genetic, biological and even psychological influences can influence the condition. As a result, obesity is a disease that eludes simple treatment or attempts to shed weight.

Weight loss is generally recommended for persons with obesity or morbid obesity. The loss of excess weight can improve the health of a person by lowering risks from obesity-related medical conditions. Methods of weight loss include dietary therapy, increased physical activity, behavior therapy, drug therapy, surgery, or a combination of therapies.

Attempts at sustained weight loss via non-surgical means within the population of the obese are overwhelmingly unsuccessful. Moreover, it is estimated that this disease has a recurrence rate in greater than 90% of cases. Consequently, long-term results of conservative treatments for obesity are generally unsuccessful, and can actually prove detrimental by producing further loss of self-esteem with the regaining of weight.

In contrast, surgery is a well-established method of long-term weight control for persons with obesity. Surgical procedures assist a person in losing weight by adjusting the way the body digests and/or absorbs calories. This is most often accomplished via surgically-implemented changes to the stomach and/or small intestine.

One general category of obesity surgery targets the relative absorption of food. This type of procedure seeks to shorten the length of, or otherwise modify, the small intestine to limit the amount of food that is ultimately absorbed by the body (malabsorption). Common examples of malabsorption procedures include: gastric bypass (e.g., Roux-en-Y gastric bypass), biliopancreatic diversion, and intestinal bypass.

Other surgical methods address obesity via restriction of food intake. This type of surgical procedure seeks to alter the size (volume) of the stomach, therefore limiting the amount of food it can hold. The result is premature satiety and a reduced intake of calories. Common examples of procedures producing food intake restriction include: vertical banded gastroplasty, gastric banding, and laparoscopic gastric banding.

Through malabsorption, food intake restriction, or some combination of both, weight is reduced since less food either enters the stomach and/or less food remains in the small intestine long enough to be digested and absorbed.

As with any surgical procedure, there are risks associated with obesity surgical procedures. Additionally, each procedure has an associated success rate that, to a certain extent, is dependent upon whether a person is willing to make certain lifestyle changes in association therewith. As a general rule, procedures that invasively alter the size or volume of the stomach carry with them increased risks, such as infections, leaking of stomach juices into the abdomen, injury to the spleen, band slippage, erosion of the band, breakdown of the staple line, and stomach pouch stretching from overeating. Such risks are due not only to the physical stapling, banding or other direct manipulation of the stomach, but also in part to the fact that the surgeon has to invade the skin, fat, fascia, muscle and peritoneum of the abdominal region to make physical contact with the abdominal cavity to conduct such procedures.

In a previously unrelated area, tissue expanders have been employed in the context of cosmetic and reconstructive surgery where the need for additional tissue is present. Tissue expanders are implantable devices capable of expansion over time. They take advantage of the fact that tissue under prolonged physical stress will stretch. Such devices are used to dissect tissue, create cavities or pockets, or separate layers of soft tissue. In use for tissue dissection, for example, a surgeon makes a remote incision into the body and inserts a hollow tissue expander into the incision to a point where a space or cavity or pocket is desired. Fluid is then forced into the expander to cause it to expand and separate two layers of tissue to form the desired space or cavity or pocket. The dissection takes place along the edges of the incision and peripherally outward from the tissue expander.

Although there have been many improvements in tissue expanders since their inception, use of tissue expanders to date has been limited to dissecting tissue, creating cavities or pockets, or separating layers of soft tissue, and the like.

There remains a need for a device and method of addressing obesity that includes only minimally-invasive surgical procedures (thus avoiding many of the associated risks of surgical procedures that invasively alter the size of the stomach), but which produce success rates in terms of weight reduction and sustainability of same comparable to current, fully-invasive surgical procedures. Ideally, such a device and related method would reduce the volume of the abdominal cavity with a minimal amount of physical invasion of the abdominal cavity.

Further, weight loss requires fat consumption. Fat cannot be reduced unless energy is consumed doing work. Obese patients are frequently unable to perform physical work to consume calories. The work of breathing has several components including compliance work which occurs with movement of the diaphragm. The diaphragm moves against intra-abdominal pressure. Surgical techniques that increase intra-abdominal pressure will force patients to increase the work of breathing, consuming calories and reducing weight. The current bariatric surgical procedures do not increase work of breathing and do not increase caloric consumption.

### SUMMARY

In general, the use of a tissue expander to produce shortening has not previously been described. A new art combines the use of an expander with a non-expandable material, where the non-expandable material is rigidly fixed to immobile tissue that retains the expander and reduces the compliance of the abdominal wall. This combination is used to reduce the convex abdominal cavity of obese patients to a linear configuration of shortened length.

The stomach is a flexible hollow organ with an average capacity of 1.5 liters within the abdominal cavity. Consuming a meal increases intragastric pressure equal to the volume consumed divided by the stomach compliance (ΔP=ΔV/C). Simultaneously the intra-abdominal pressure increases equal to the volume consumed divided by the abdominal wall compliance. Satiety is usually achieved without filling the stomach to capacity and the compliance of the stomach is greater than the compliance of the anterior lateral abdominal wall. Therefore, the pressure increase within the abdominal cavity is greater than the pressure increase within the stomach and the flexible stomach collapses to equalize the pressure. Satiety pressure is generated by the meal volume within the abdominal cavity not from the pressure generated by the meal volume within the stomach. Reducing the compliance of the abdomen wall will produce the same physiology effects as reducing the compliance of the stomach. Abdominal wall surgery is less evasive and safer than the current intra-abdominal bariatric surgical procedures.

The present disclosure describes devices and methods for controlling body weight via indirect reduction of the volume of the abdominal cavity. One embodiment of the present disclosure includes a device for increasing intra-abdominal pressure from a point superficial to the fascia of the abdominal cavity to create satiety by decreasing the volume of the abdomen without physically invading the abdominal cavity. In an embodiment of the method of the present disclosure, an incision is made in the abdominal region of the body. Next, a restriction device is placed into the incision to a point superficial to the fascia of the abdominal region, and connected to said point via coupling or attachment means for connecting the restriction device to the fascia. The presence of the restriction device reduces the compliance of the abdominal wall, increases intra-abdominal pressure, reduces the volume of the abdominal cavity and more quickly creates satiety than would occur without the restriction device.

One embodiment of the restriction device is passive in nature. Upon placement, the passive restriction device decreases abdominal wall compliance, increases intra-abdominal pressure, reducing the volume of the abdominal cavity and thus reducing the amount of food required to achieve satiety. In alternative embodiments, one or more additional passive restriction devices can be used to supplement the effects of the initially-placed passive restriction device. In those embodiments, a second passive restriction device is placed into and attached to a point within the abdominal wall (e.g., to overlap the initial passive restriction device) to further increase the exerted intra-abdominal pressure and further reduce the volume of the abdominal cavity. In some instances, the second passive restriction device may be added at the same time (i.e., during the same procedure) as the first passive (or expandable) restriction device. In others, additional passive restriction devices may be added at a later time (i.e., during a second or additional procedure) as certain weight loss plateaus or goals are reached, or where weight loss has not progressed or is not sufficiently progressing with the initial or prior-placed restriction device(s) alone. More than one additional passive restriction device may be inserted after the insertion of the initial passive (or expandable) restriction device, particularly after periodical reassessment of weight loss due to the initial or previously-inserted restriction device(s) and the weight loss' associated effects on the body, as well as to gradually introduce additional intra-abdominal pressure.

Another embodiment of the restriction device is capable of being expanded subsequent to implantation. Following placement and attachment (or coupling) to the point superficial to the fascia of the abdominal cavity, the expandable version of the restriction device (or expandable restriction device) is expanded as needed to maintain intra-abdominal pressure. Intra-abdominal pressure is determined by the compliance of the passive component. After the passive component is placed the intra-abdominal pressure is increased and the patients eat less to achieve satiety. As a result, intra-abdominal fat is consumed and intra-abdominal volume decreases. The expandable portion of the device is filled to compensate for the volume lost by fat consumption. The expandable portion is inflated to maintain the intra-abdominal pressure generated by the compliance of the passive component. The expander does not expand the abdominal cavity but functions to compensate for fat volume loss and transferring the pressure generated by the passive component to the abdominal cavity. In fact, the expander acts to decrease the volume of the abdominal cavity.

Using either the passive or expandable version of the restriction device, placement and use of a restriction device increases the intra-abdominal pressure, effectively reducing the volume of food needed by the person to achieve satiety. The ingestion of less food by the person will also necessarily advantageously affect relative food absorption. Both contribute to weight loss. Importantly, such intra-abdominal pressure is created by either version of the restriction device without physical invasion of the abdominal cavity. In addition to increased intra-abdominal pressure, both devices decrease the compliance of the abdominal wall. In some instances, the devices may be considered to reverse the effects of the stretching or damage done to the abdominal cavity due to genetics, age, nutrition, pregnancy, and lack of physical fitness. Specifically, tension generated by continuous or prolonged stretching of the abdominal muscles and fascia causes the abdominal wall in general to slowly become more compliant, or elastic, over time, until weakening or total failure of the abdominal wall occurs. Weakening or failure of the abdominal wall, fascia, and/or the muscles within the abdominal wall increases the compliance of the abdominal wall and increases the available volume in the abdominal cavity based on the abdominal wall's increased compliance. The stretching, damage, and/or failure of the abdominal wall occurring based on any of the above effectively increases the volume of food needed by the patient to achieve satiety due to the abdominal wall's increased compliance and abdominal cavity volume. The use of either type of device described in the present disclosure can be used to decrease the compliance of the abdominal wall, and may reduce or prevent said stretching from occurring, while also assisting the reconstruction of the abdominal wall after failure or severe weakening has occurred. Further, and directly associated with both the increase in intra-abdominal pressure and the decrease in the compliance of the abdominal wall, the device causes an increased work of respiration in patients after insertion, as the patient's diaphragm must return to its original position prior to device placement. The devices require the diaphragm to create greater pressure to oppose the increased intra-abdominal pressure, thus resulting in more work required for breathing. The additional work required and the increased post-operative demand for oxygen leads directly to the consumption of additional calories, causing increased energy expenditure, fat consumption, and weight loss.

An embodiment of the expandable version of restriction device is an expandable hollow member including means for expansion and contraction, as well as coupling or attachment means for coupling or attaching the device to an appropriate portion of the body (e.g., a point superficial to the fascia of the abdominal cavity). Optimally, the hollow member includes both a rigid and a flexible region along its exterior surface. This feature, when the hollow member is oriented properly, attached to the body in the appropriate location and manner (e.g., at the proper point superficial to the fascia of the abdominal cavity), and expanded via the means for expansion, will direct pressure towards the abdominal cavity. Such pressure will effectively reduce the volume of the abdominal cavity, causing the person to require less food to achieve satiety. The expandable version of the restriction device may be effectively designed or created as a one- or two- part device. In the one-part embodiment of the device, the expandable hollow member is integral (or connected) to the rigid and flexible regions along the hollow member's exterior surface. The rigid portion of the hollow member may continue past the general surface of the hollow member, as well as include the coupling means or members. The rigid portion may then be used to attach the device to the appropriate portion of the body such that when the expandable hollow member is expanded via the means for expansion, the rigid portion of the device remains relatively stationary without expanding, such that the pressure from the expansion is directed towards the abdominal cavity, the opposite direction from the rigid portion of the hollow member.

In the two-part embodiment of the device, the expandable hollow member may be a separate component from the rigid portion of the device. For instance, the expandable hollow member may be a tissue expander that is positioned at an appropriate location within the body (e.g., at a point superficial to the fascia of the abdominal cavity), while the separate, rigid portion of the device is a surgical mesh or other suitable component (e.g., medical grade plastic, nylon, polyester, and even metal such as stainless steel) that is placed or positioned above the tissue expander. The surgical mesh (or other substitute component) may then be attached to the body such that the tissue expander remains stationary in the appropriately selected location, both during and after expansion and/or contraction of the expandable hollow member. When the hollow member is expanded, the relatively flexible portion of the hollow member (optimally positioned opposite the surgical mesh from the abdominal cavity) expands and maintains and transmits pressure directed to the abdominal cavity.

Once in place using the appropriate method, the level of expansion of the one- or two-piece embodiment of the expandable version of the restriction device (hollow member) can be selectively adjusted via the means for expansion without the need for additional surgery. If, for example, weight loss is occurring too rapidly for the person, the expansion can be reduced. The effect of the reduction of expansion will be a reduction of intra-abdominal pressure allowing the person to ingest a higher volume of food. If, on the other hand, weight loss is not progressing or has reached a plateau, expansion of the device can be increased, thereby increasing the intra-abdominal pressure and further reducing the volume of food needed by the person to achieve satiety. Further, once a certain level of weight loss has been reached, a second operation may be performed to remove the hollow member and, in some instances, permanently or semi-permanently attach or secure the relatively rigid or non-expandable portion of the device (or add a new, and in some cases, separate, rigid portion) to the appropriate position within the abdominal cavity to ensure that the intra-abdominal pressure and cavity size at the time of the expandable portion's removal remains relatively constant, even after removal of the expandable hollow member, so that the results achieved from the device with the hollow member remain even after the hollow member has been removed. In some instances, the device may then be considered a passive restriction device, in that the remaining portion of the device is no longer expandable, while still providing the effects of the decreased volume in the intra-abdominal cavity.

The details of one or more embodiments of the present disclosure are set forth in the accompanying drawings and the description below. Other features, objects, and advantages of the present disclosure will be apparent from the description and drawings, and from the claims.

### DESCRIPTION OF DRAWINGS

FIG. 1A is a cross-section of an abdominal cavity of a human illustrating normal anatomy of same;

FIG. 1B is a cross-section of an abdominal cavity of a human illustrating obese anatomy of same;

FIG. 2A is a cross-section of an abdominal cavity of a human showing an obese anatomy and including an embodiment of a one-piece expandable version of the restriction device of the present disclosure in a non-expanded state;

FIG. 2B is a cross-section of an abdominal cavity of a human showing an obese anatomy and including an embodiment of the two-piece expandable version of the restriction device in the present disclosure in a non-expanded state;

FIG. 2C is a cross-section of an abdominal cavity of a human showing an obese anatomy and including an embodiment of the passive version of the restriction device in the present disclosure;

FIG. 2D is a top (or frontal) view of an abdominal cavity of a human showing an obese anatomy and including an embodiment of the expandable version of the restriction device in the present disclosure;

FIG. 3A is a flow chart outlining the steps of an embodiment of the method of the present disclosure employing an expandable restriction device;

FIG. 3B is a flow chart outlining the steps of an embodiment of the method of the present disclosure employing one or more passive restriction devices;

FIG. 4A is a cross-section of an abdominal cavity of a human showing an obese anatomy and including an embodiment of the one-piece expandable version of the restriction device in an expanded state;

FIG. 4B is a cross-section of an abdominal cavity of a human showing an obese anatomy and including an embodiment of the two-piece expandable version of the restriction device in an expanded state; and

FIG. 4C is a cross-section of an abdominal cavity of a human showing an obese anatomy and including an embodiment of the present disclosure applying multiple passive restriction devices.

### DETAILED DESCRIPTION

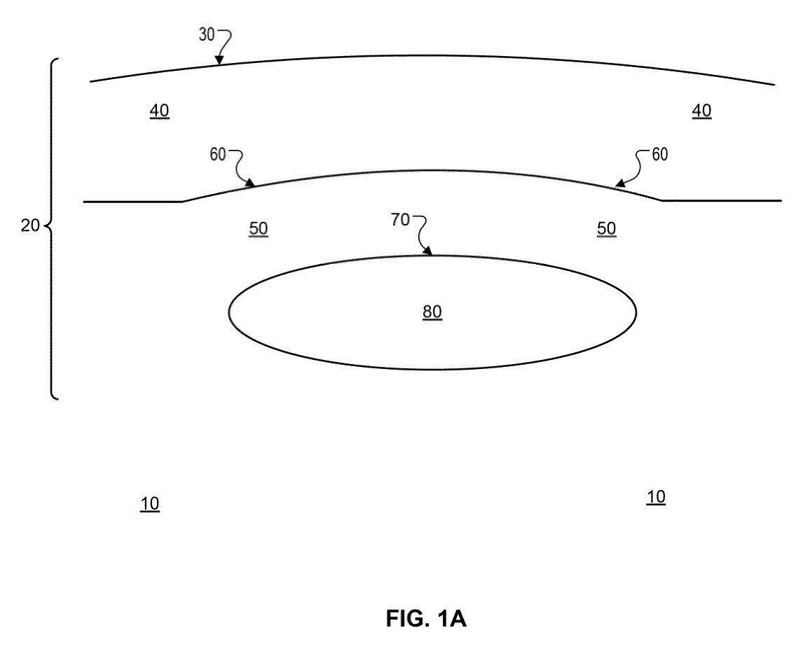

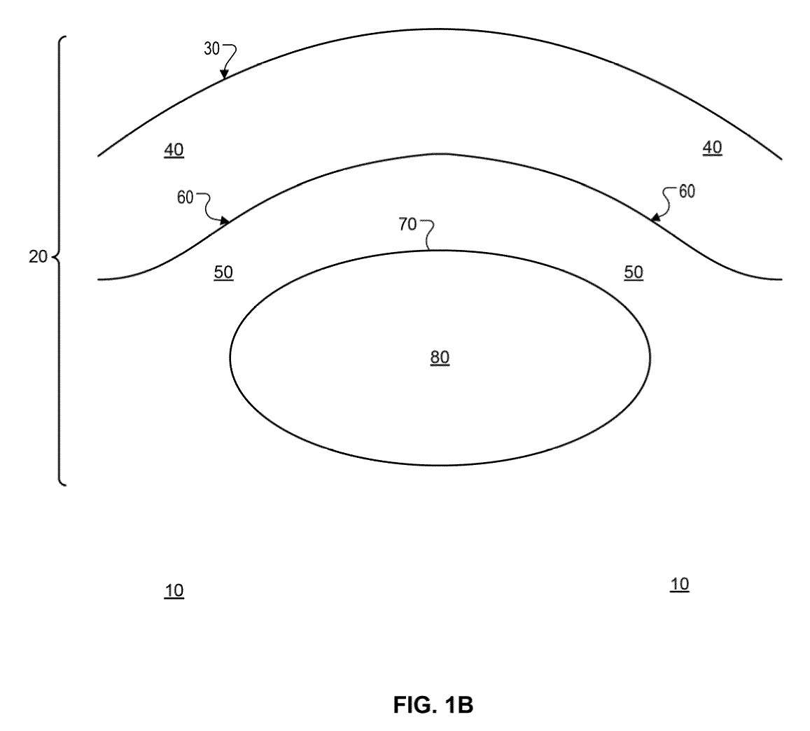

The present disclosure comprises a device and method for controlling body weight via indirect reduction of the volume of the abdominal cavity. FIG. 1A, a cross-section of an abdominal cavity of a human exhibiting normal anatomy, illustrates a body 10, said body including an abdominal region 20 having skin 30, fat 40, muscle 50, fascia 60, a peritoneum 70, and an abdominal cavity 80. The abdominal cavity 80 comprises the stomach (not shown) which receives and processes food and other nourishment for the body 10, passing same to the intestines (not shown) of the body 10.

FIG. 1B is a cross-section of an abdominal cavity of a human exhibiting an obese anatomy, also including an abdominal region 20 having skin 30, fat 40, muscle 50, fascia 60, a peritoneum 70, and an abdominal cavity 80. As illustrated, the enlarged abdominal cavity 80 of the body 10 in FIG. 1B causes, over time, an increased compliance in the abdominal wall, and particularly the fascia 60. The increased compliance of the abdominal wall and the corresponding increase in the available volume of the abdominal cavity combine (along with other factors, such as age, injury, etc.) to cause an increase in the volume of food needed by the patient to achieve satiety. Because of the increased volume of food necessary to be satisfied, obesity is a common effect. Further, in response to the increased size of the abdominal cavity 80 and the increased compliance of the abdominal wall, the skin 30 of the obese anatomy is formed in a much more convex in shape than that of the normal body illustrated in FIG. 1A.

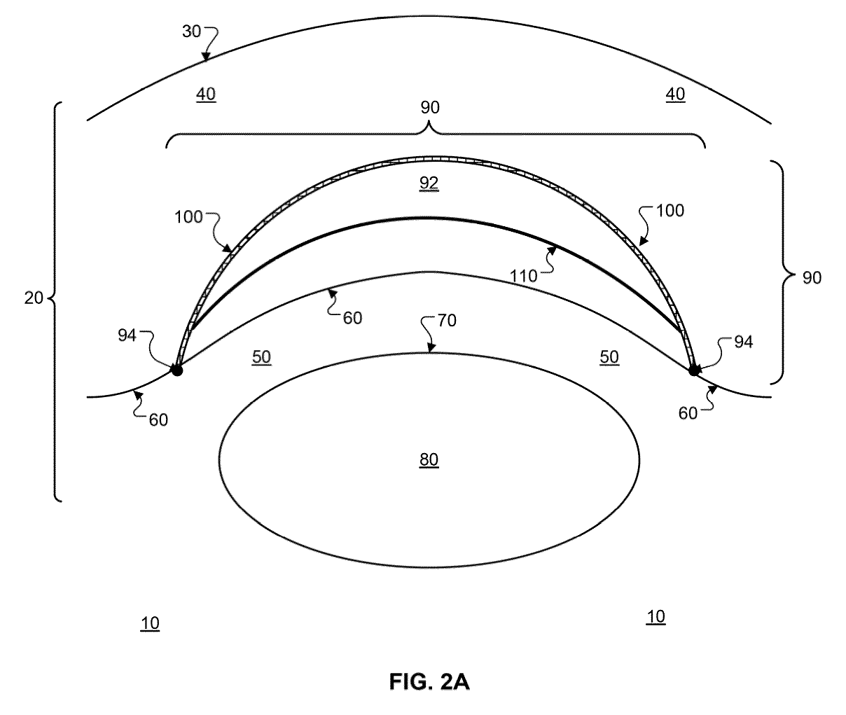

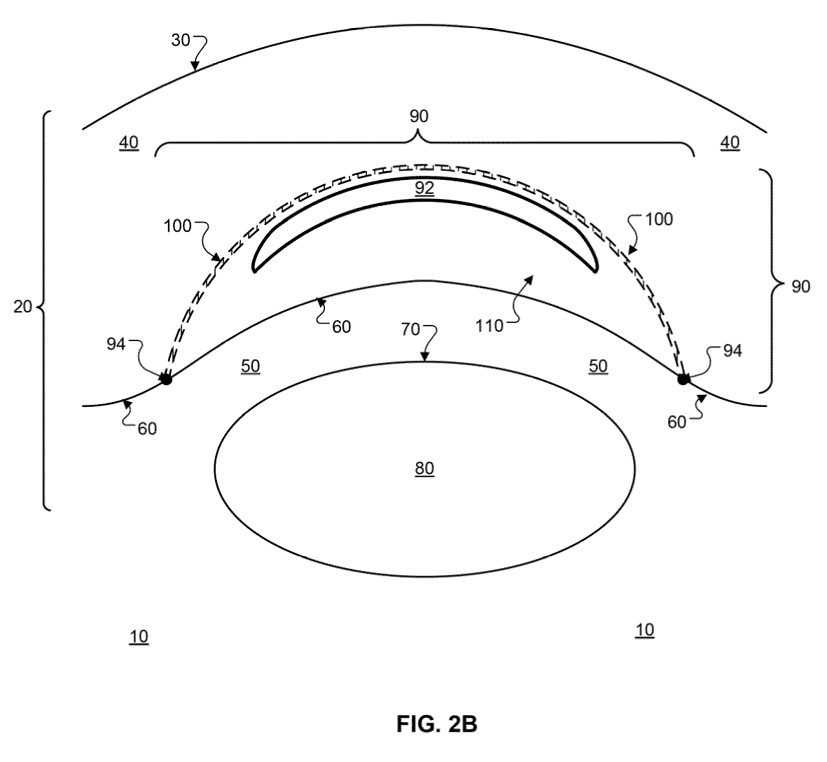

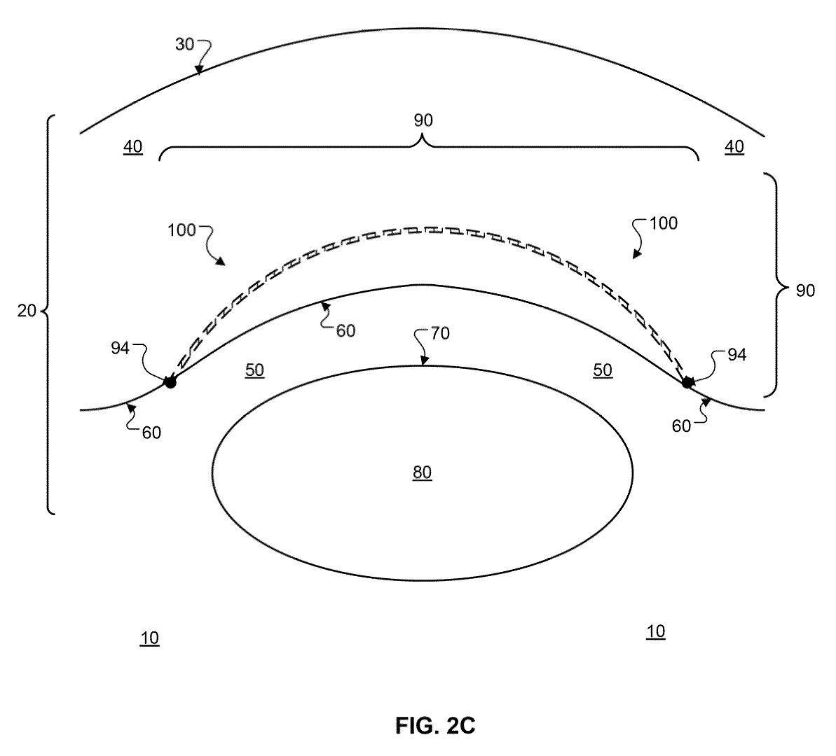

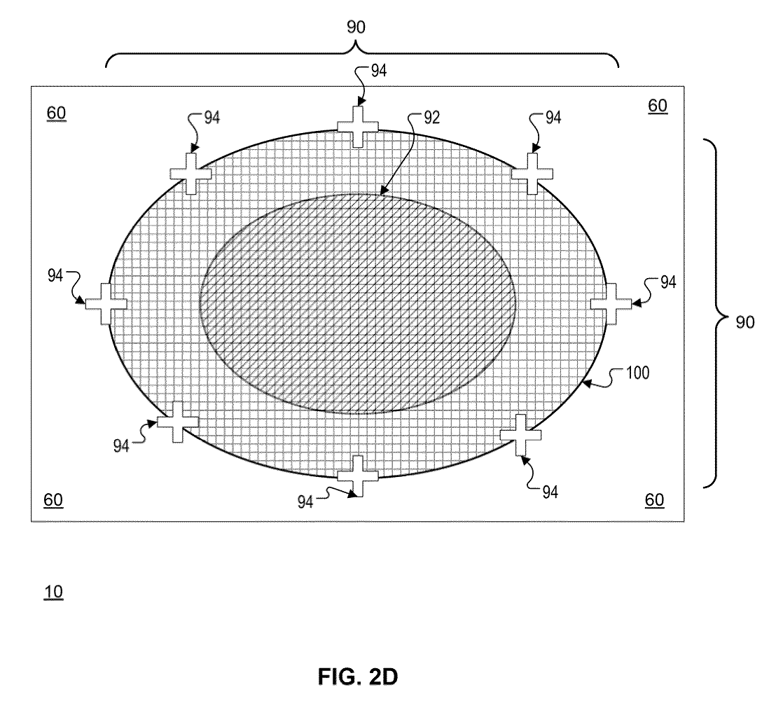

FIG. 2A illustrates a cross-sectional view of the abdominal region 20 of the obese body 10 including placement and attachment of a one-piece embodiment of an implantable food restriction device 90 of the present disclosure in its non-expanded mode. This version of the implantable food restriction device 90 is preferably comprised of a hollow member 92, means for expansion and contraction (not shown) of the hollow member 92, and coupling or attachment means 94 for coupling or attaching the device to a portion of the abdominal region 20. The hollow member 92 preferably includes a relatively non-expandable portion 100 and a relatively expandable portion 110.

In some instances, such as the one illustrated in FIG. 2A, the relatively non-expandable portion 100 of the hollow member 92 may continue past the general boundaries or edge of the hollow member 92 (i.e., may extend past the general circumference of the hollow member 92), such that the relatively non-expandable portion 100 can come into contact with points within or superficial to the abdominal wall, such as to the fascia 60 as illustrated in FIG. 2A. Further, the relatively non-expandable portion 100 may include coupling or attachment means 94 for coupling the non-expandable portion 100 (and therefore the restriction device 90) to a portion of the body in order to constrict or contain the hollow member 92 in the appropriate position. This position may differ based on the characteristics of the particular body in which the device 90 is being placed. In particular instances, the coupling or attachment means 94 may include surgical sutures, surgical staples, a medical spring coil (causing the non-expandable portion 100 to attach itself to the appropriate abdominal wall location), a tissue adhesive (e.g., a medical grade glue suitable for internal adhesions) capable of forming a bond between the non-expandable portion 100 of the device 90 and the selected portion superficial to the abdominal cavity 80, or any other appropriate coupling or attachment means. Additionally, the non-expandable portion 100 may itself be capable of forming a natural bond to the fascia 60. In general, the coupling or attachment means 94 acts to rigidly fix the relatively non-expandable portion 100 of the device 90 to the abdominal wall in order to minimize, and in some cases, eliminate movements or shifting of the device 90 during use, as well as to maintain a level of intra-abdominal pressure from insertion and attachment going forward.

In some instances, the coupling or attachment means 94 may be part of the non-expandable portion 100 of the device 90, while in other instances the means 94 may be a separate portion of the device 90 used for coupling, attaching, or otherwise connecting the non-expandable portion 100 to the selected portion superficial to the abdominal cavity 80. Still further, the coupling or attachment means 94 may be located along the exterior portion of the non-expandable portion 100 such that the device 90 may be attached in multiple locations. In some instances, the coupling or attachment means 94 may be located or attached continuously along the edges of the non-expandable portion 100 so that the device 90 can be circumferentially coupled to the selected position within the body. This type of coupling may be accomplished using surgical sutures to connect the outside edges of the non-expandable portion 100 as well as through the use of a tissue adhesive along the outside edges of the non-expandable portion 100. In other instances, the coupling or attachment means 94 may be located periodically along the edges of the non-expandable portion 100 of the device 90, providing for a finite number of connections between the device 90 and the abdominal wall (e.g., the fascia 60). While not continuous, the number of periodic means 94 used should be sufficient to constrict the device 90 to the selected location during routine and non-routine, but anticipated movements associated with the body 10. Surgical staples or other similar methods of attachment to the abdominal wall used during surgery may be examples where periodic attachment is applied.

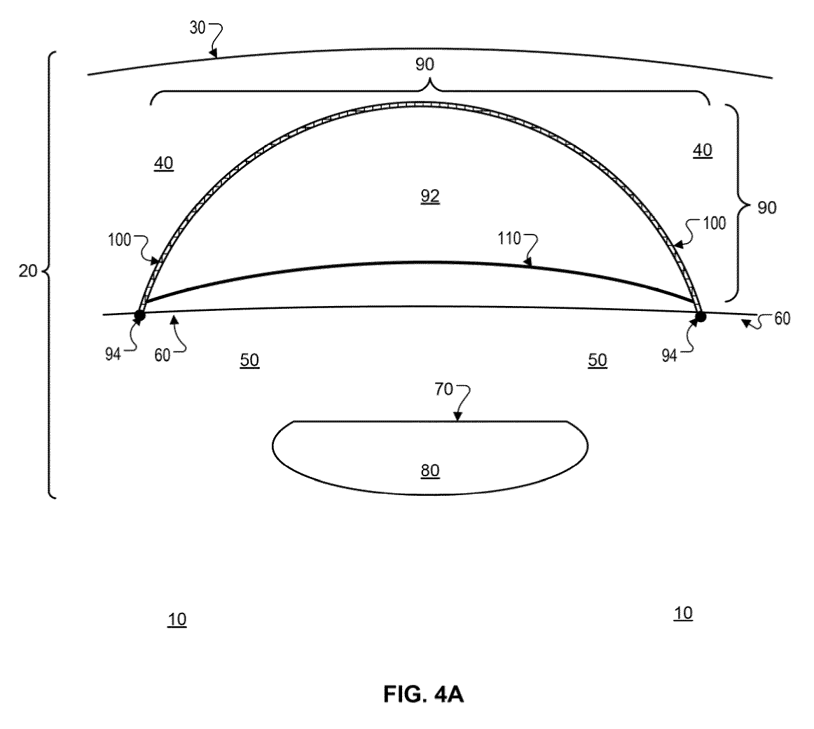

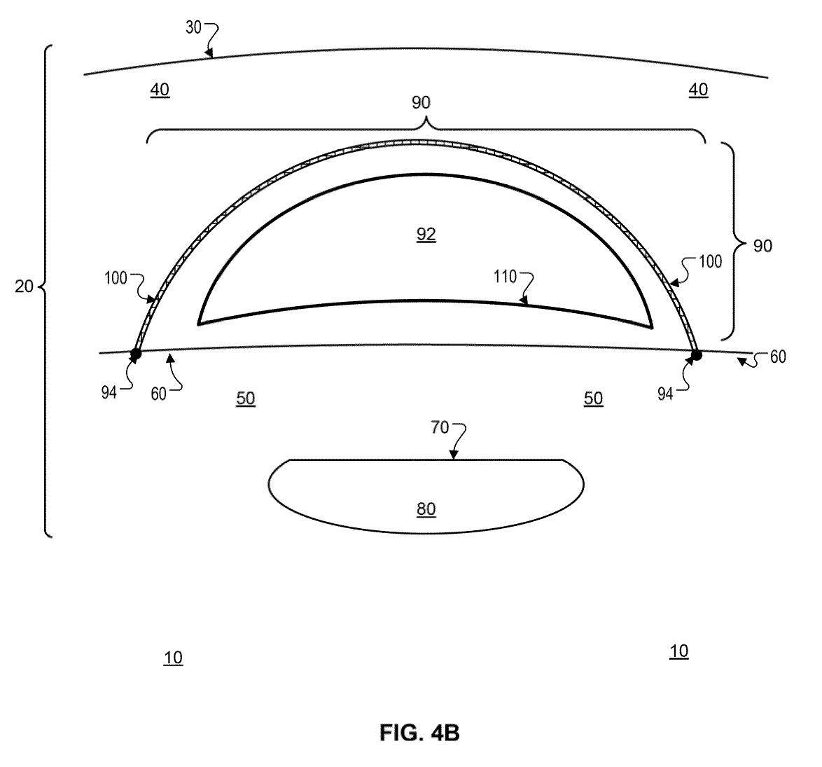

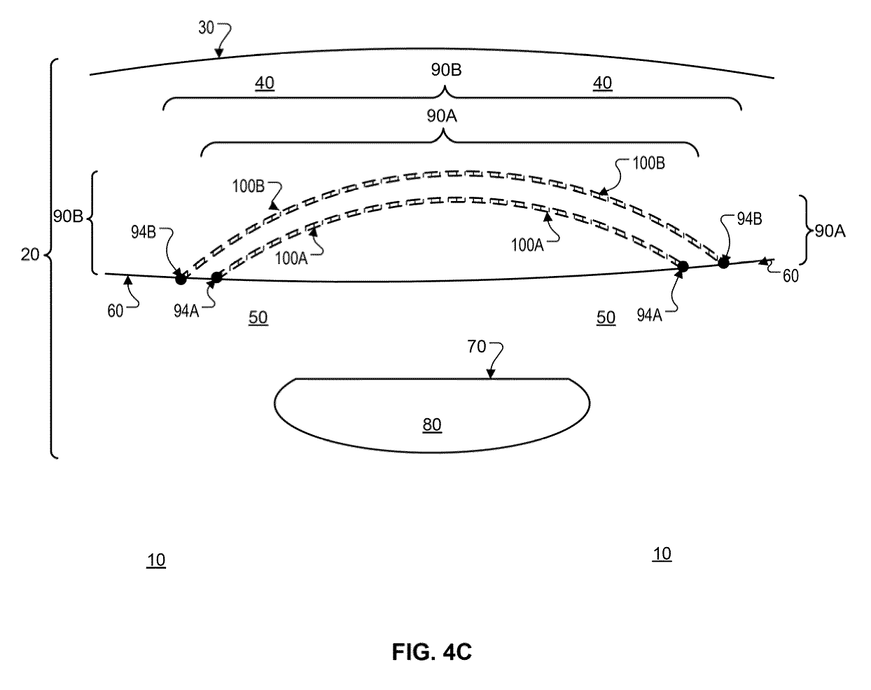

When the device 90 is placed in the abdominal region 20 of the body 10, the relatively expandable portion 110 of the device 90 is oriented towards the abdominal cavity 80, thereby positioning the relatively non-expandable portion 100 of the hollow member 92 away from the abdominal cavity 80 (i.e., towards the fat 40). Using the coupling or attachment means 94, the relatively non-expandable portion 100 of the device 90 is attached to the body in order to securely fasten or rigidly fix the device 90 (and, specifically, the hollow member 92) to the position selected. In most instances, the ideal location for connecting the device 90 to the abdominal wall is at a position superficial to the fascia 60. Once placed within the abdominal region 20, coupled to the abdominal wall (e.g., the fascia 60) via the coupling or attachment means 94 of the non-expandable portion 100, and expanded (see FIG. 4A), the hollow member 92 will apply the force towards the abdominal cavity 80, thereby reducing the relative volume of the abdominal cavity 80. Although the hollow member 92 of the implantable food restriction device 90 of this embodiment is described as having a relatively expandable portion 110 and a relatively non-expandable portion 100, it is noted that the implantable food restriction device 90 can utilize a hollow member 92 lacking the relatively expandable portion 110 and/or the relatively non-expandable portion 100. Any expansion of the implantable food restriction device 90 will transfer pressure created by the compliance of the non-expandable portion that will reduce the relative volume of the abdominal cavity 80. Therefore, any suitable design of the implantable food restriction device 90 is contemplated by this disclosure.

FIG. 2B illustrates a cross-sectional view of the abdominal region 20 of the body 10 including placement and attachment of the two-piece, expandable embodiment of an implantable food restriction device 90 of the present disclosure in non-expanded mode. This version of the implantable food restriction device 90 is preferably comprised of a hollow member 92, means for expansion and contraction (not shown) of the hollow member 92, a relatively non-expandable component 100 separate from the hollow member 92 used to maintain the hollow member 92 in a selected position adjacent to the abdominal wall, and coupling or attachment means 94 for coupling the relatively non-expandable component 100 to a portion of the abdominal wall surrounding the hollow member 92. The hollow member 92 illustrated in FIG. 2B includes a relatively expandable portion 110 that can be expanded and contracted via the device's 90 means for expansion and contraction. For example, the hollow member 92 may be a tissue expander or other expandable component that is positioned at a point within the abdominal wall (i.e., the location within the abdominal wall determined to provide the optimal or necessary intra-cavity pressure towards abdominal cavity 80 when the hollow member 92 is expanded). The relatively non-expandable component 100 can then be placed on top of the hollow member 92 (i.e., opposite the hollow member's 92 connection to the point superficial to the abdominal cavity 80) and attached to points superficial to the abdominal cavity 80 (e.g., the fascia 60), forming a pocket in which the hollow member 92 can be expanded. In some instances, the relatively non-expandable component 100 comprises a relatively non-expandable material, such as medical grade polypropylene mesh, that can be used to restrict the hollow member 92 within the created pocket and in the position at which the hollow member 92 is originally placed. Specifically, the relatively non-expandable component 100 of FIG. 2B may be attached or coupled to the abdominal wall (i.e., the fascia 60, as shown) via the coupling or attachment means 94 in an area surrounding the hollow member 92, as illustrated in FIG. 2B. In some instances, the same components as those described with regard to the one-piece device of FIG. 2A may be used for the device of FIG. 2B, with the distinction being that the relatively non-expandable component 100 of FIG. 2B is not integral, or physically connected to, the hollow member 92 as in the device of FIG. 2A.

Because the expandable hollow member 92 is positioned beneath the relatively non-expandable component 100 (e.g., between the non-expandable component 100 and the fascia 60), the expansion of the hollow member 92 will be restricted by the non-expandable component 100, such that the force of pressure created when the hollow member 92 is expanded will be applied towards the fascia 60, the peritoneum 70, and, ultimately, the abdominal cavity 80, thereby reducing the relative volume of the abdominal cavity 80. The hollow member 92 may be constructed such that without the non-expandable component's 100 restriction, the hollow member 92 would expand equally in every direction when the means for expansion are used. In other instances, however, the hollow member 92 may be constructed such that expansion causes a particular side or portion of the hollow member 92 to expand relatively more rapidly than another side or portion of the hollow member 92 (e.g., the bottom portion of the hollow member 92 directed towards the fascia 60 as illustrated in FIG. 2B may expand at a slower rate than the top portion directed towards the non-expandable component 100). While it may be advantageous to position the portion of the hollow member 92 that expands more slower toward the abdominal cavity 80, the secure attachment (i.e., rigid fixation) of the relatively non-expandable component 100 to the body (i.e., the fascia 60) insures that any expansion of the hollow member 92 will cause a force to be applied towards the abdominal cavity 80 based on the relatively non-expandable component's 100 restriction upon the hollow member's 92 expansion. In the preferred embodiment, the relatively non-expandable component is rigidly fixated (e.g., securely attached or fastened) to the body via the coupling or attachment means 94 in order to produce a shortening of the fascia 60, and circumferentially attached around the hollow member 92 in order to prevent displacement of the member 92 after the hollow member's 92 positioning within the body. The rigid fixation of the device 90 to the abdominal wall ensures both the positioning of the device 90 in general and the maintenance of the desired intra-abdominal pressure exerted towards the abdominal cavity.

One embodiment of the expandable version of the implantable food restriction device 90 includes means for expansion and contraction (not shown). Such means for expansion and contraction can be any suitable means for providing and extracting a liquid or gas from the hollow member in a manner that does not require additional surgical procedures. For example, one such means is a tube that is relatively hollow and flexible and which protrudes from, or is contained within, the body when attached to the hollow member 92 of the implantable food restriction device 90. In some instances, the tube may be positioned over a bone or other relatively accessible area within the body so that expansion and contraction procedures can be performed without substantially invading the body. The tube is utilized to provide and extract liquids or gases from the hollow member 92. If a liquid or gas is provided to the hollow member 92, it expands and reduces the relative volume of the abdominal cavity 80.

In the expandable versions of the implantable food restriction device 90, the intra-abdominal pressure created by the compliance of the non-expandable component of the implantable food restriction device 90 can be controlled over time by providing and/or extracting a liquid or gas from the hollow member 92 in response to weigh loss progress of the body 10. Although a tube means has been described herein, it is noted that any suitable means for providing/extracting a liquid or gas from the hollow member 92 of the implantable food restriction device 90 may be employed with the devices and methods described herein. For example, the hollow member 92 may include a magnetic injection port or the like, allowing the port (and device) to be located without the need for an invasive procedure, internal imaging of the body, or other normally required techniques. Once the magnetic injection port is located (e.g., by use of a magnet), a syringe, needle, or other device may penetrate the skin 30 and fat 40 to access the hollow member 92, allowing for the expansion or contraction of the hollow member 92 without the need for a relatively more invasive procedure. Additionally, the compliance of the non-expandable component 90 will result in additional pressure towards the abdominal cavity, and in response, a loss in weight and intra-abdominal fat (and therefore volume) will occur. The hollow member 92 can then be expanded in order to replace, or compensate, for said loss volume. For example, as the abdominal wall flattens because of the device 90, the rigidly fixated, relatively non-expandable component 100 may exert less pressure on its own. Thus, the expansion of the hollow member 92 can be used to compensate for said loss of pressure by filling the additional volume, thus maintaining or returning to the decreased level of compliance abdominal wall initially generated due to the rigid fixation of the non-expandable component 100.

FIG. 2C illustrates a passive version of the implantable food restriction device 90. The implantable food restriction device 90 can effectuate weight loss as a passive member, without the need for any expansion when implanted. For example, either of the devices illustrated in FIGS. 2A or 2B could be used as a passive device by not inflating the hollow member 92 provided in both devices. The mere presence of a passive member, such as the implantable food restriction device 90 illustrated in FIG. 2C, will create a force that is applied towards the abdominal cavity 80. Like with the expandable version of the implantable food restriction device 90, the passive version will create intra-abdominal pressure that will reduce the relative volume of the abdominal cavity 80, thereby reducing the relative food intake associated with satiety.

FIG. 2C illustrates a purely passive food restriction device 90 comprising a relatively non-expandable component 100 and coupling or attachment means 94. In some embodiments, the passive food restriction device 90 may also include an optional hollow member 92 located between the relatively non-expandable component 100 and the abdominal cavity 80 (e.g., between the relatively non-expandable component 100 and the fascia 60). The passive version of the implantable food restriction device 90 illustrated in FIG. 2C may be constructed of any appropriate materials, such as medical grade polypropylene mesh or the like. Additionally, the relative size and dimensions of the passive version of the relatively non-expandable component 100 and/or the implantable food restriction device 90 may be modified to vary the intra-abdominal pressure created from the use of same, as well as to fit the size and medical needs of the particular patient in which the device 90 is to be inserted. Although a polypropylene mesh is described above, it is noted that the passive implantable food restriction device 90 may be constructed of any appropriate material or materials, including medical grade plastics, nylon, polyester, and even metals (stainless steel). Similar to the expandable versions of FIGS. 2A and 2B, the passive restriction device 90 is securely attached to the body within the abdominal region 20 by the device's 90 coupling or attachment means 94. The specific means used to attach the device may be the same or different than those described in association with the expandable restriction devices.

The versions of the food restriction device 90 of the present disclosure can be constructed of any suitable material(s), and are preferably constructed of a durable, relatively flexible material or materials capable of being safely used for prolonged periods of time within the human body, such as surgical grade plastics, polymers, and the like. Additionally, both the passive and expandable versions of the food restriction device 90 may be sized and shaped in any suitable combination to produce the desired level of intra-abdominal pressure necessary to achieve desired weight loss by the body 10. It is noted that varying sizes, shapes, and combinations thereof of the food restriction device 90 may be employed as suggested by the individual needs of a particular body 10 to optimize the results achieved through use of the devices and methods disclosed herein. For example, while the non-expanded hollow member 92 is generally illustrated in a half-moon, and oblong shape, it may be designed in any suitable manner to achieve the desired results (e.g., relatively spherical, shaped specifically for the body of each patient).

FIG. 2D provides an illustration from a perpendicular (or top) view into the abdominal region of a body of a particular embodiment of the restriction device 90. As shown, the device 90 is positioned at a point superficial to the fascia 60 within the body 10. Specifically, the hollow member 92 is placed at a particular, selected point on the fascia 60, and the relatively non-expandable portion (in the one-piece expandable embodiment) or component (in the two-piece expandable embodiment or the passive embodiment) 100 is placed above the hollow member 92 in order to restrain the hollow member 92 in the selected position. In the particular embodiment illustrated in FIG. 2D, the non-expandable portion/component 100 is attached to the fascia 60 via eight (8) individual coupling or attachment means 94 located around the centrally located hollow member 92. Although illustrated as attached to the fascia 60 at eight individual locations 94, the relatively non-expandable portion/component 100 of the device 90 may be, in some embodiments, continuously attached to the fascia 60 along the periphery of the non-expandable component 100 (i.e., around the entire edge of the component 100 touching or associated with the fascia 60), or may be attached via any other appropriate number of attachment means 94. In some embodiments, the optimal method for fastening the means for coupling or attachment 94 may be circumferentially around the hollow member 92, in order to ensure that the hollow member 92 remains in place during expansion and contraction of the device 90, as well as during any movement of the body 10 or abdominal cavity.

FIG. 2D may also represent a passive device 90, wherein the hollow member 92 is not expanded or contracted, but instead is used passively to provide the intra-abdominal pressure necessary to reduce the volume of the intra-abdominal cavity 80 for weight loss purposes. Additionally, certain passive devices 90 may be provided as shown in FIG. 2D, but without the hollow member 92. In those instances (such as that illustrated in FIG. 2C), surgical mesh or some other restrictive material may be used alone to create the desired effects of the present disclosure.

In general, the result of exerting pressure on the abdominal cavity is to control the relative volume of same. Increased pressure in the abdominal cavity will reduce its relative volume, causing the body (e.g., person) to require less food to achieve satiety while also increasing the work of breathing, thus consuming additional calories. The ingestion of less food by the body will also necessarily advantageously affect relative food absorption. Importantly, the hollow member of the expandable version of the implantable food restriction device can be expanded or contracted without the need for additional surgery, allowing for highly flexible control over weight gain for the body. If, for example, weight loss is occurring too rapidly for the body, the expansion of the device (i.e., the hollow member) can be reduced. The effect of the reduction of expansion will be a reduction of intra-abdominal pressure created by same, allowing the person to ingest a higher volume of food (and also adjusting the relative food absorption rate in the lower intestine). The overall result will be a slowdown in weight loss experienced by the body. If, on the other hand, weight loss is not progressing at a satisfactory rate (e.g., weight loss has reached a plateau, or additional weight loss is desired), the implantable food restriction device 90 can be expanded, thereby increasing the intra-abdominal pressure created by the device, reducing the volume of food needed to achieve satiety and advantageously affecting the relative food absorption rate. Alternatively, where a passive version of the device is used, additionally passive devices or the use of expandable devices may be added to the body in order to incrementally increase the relative intra-abdominal pressure exerted by the collection of devices. While a second surgery to insert the additional passive or expandable devices may be necessary, the situations and preferences of particular patients, physicians, and/or surgeons may make such an option more desirable than the expandable version of the device.

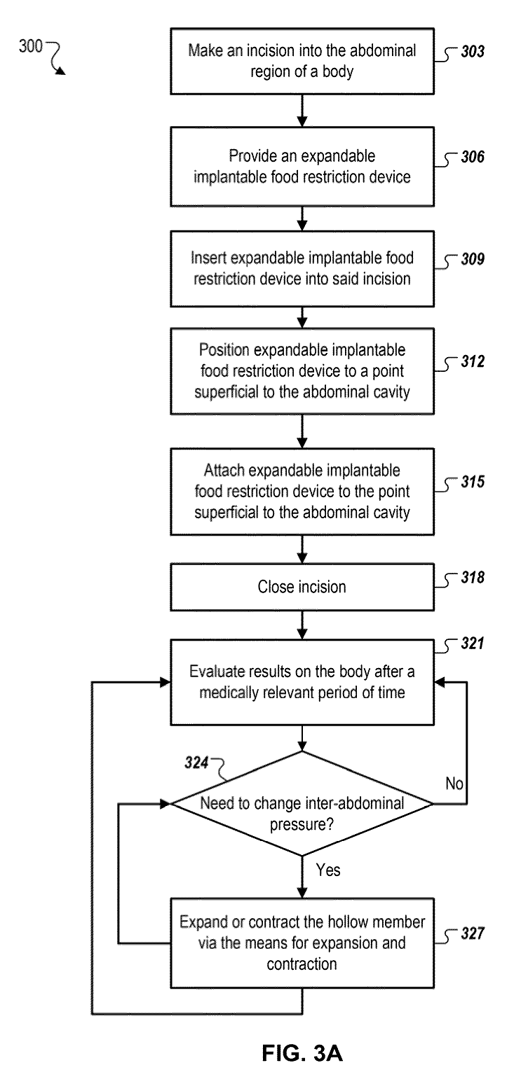

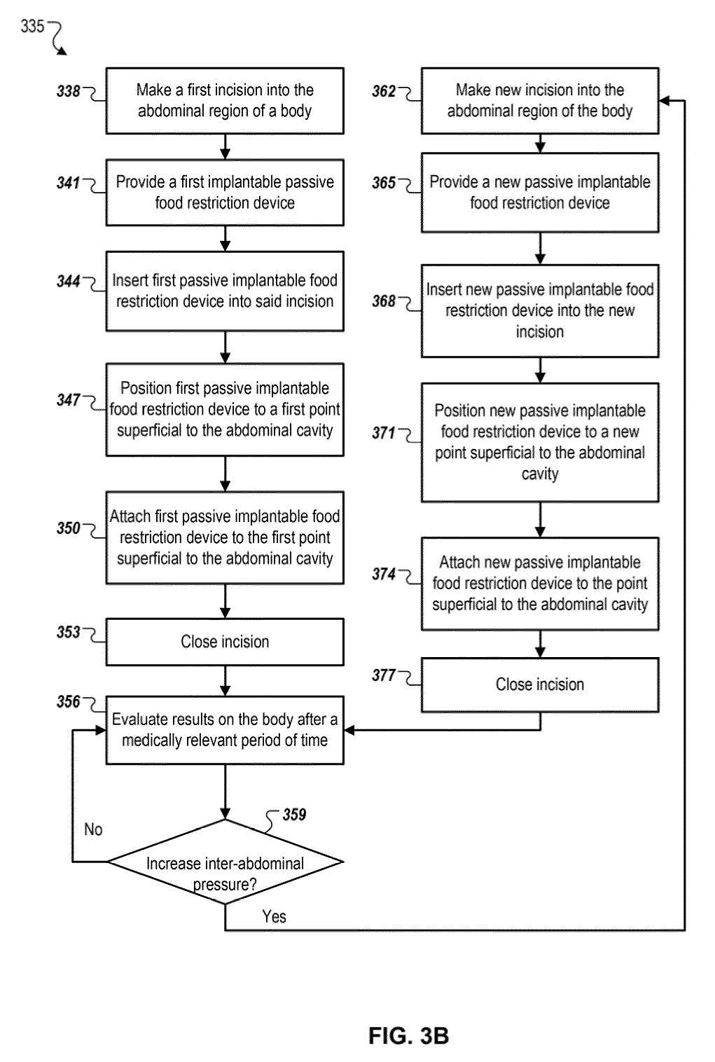

Surgical methods for controlling weight of a body using the devices described herein are illustrated in FIGS. 3A and 3B. FIG. 3A illustrates a method 300 associated with an expandable embodiment of the device (such as those described in FIGS. 2A and 2B), while FIG. 3B illustrates a method 335 associated with passive embodiments of the device (such as that described in FIG. 2C).

FIG. 3A illustrates one embodiment of a surgical method 300 for controlling the weight of a body using an expandable version of the present device, said body including an abdominal region having skin, fat, muscle, fascia, a peritoneum and an abdominal cavity. At 303, an incision is made into the abdominal region of a body. The incision can be made utilizing any traditional means for same, including via scalpel, laser, or other suitable cutting device. At 306, an implantable food restriction device is provided, said implantable food restriction device comprising an expandable version of the device (e.g., a hollow member and associated means for expansion and contraction). The particular version of the expandable device may be a one- or two-piece device, wherein the one-piece device is described with regard to FIG. 2A, while the two-piece device is described with regard to FIG. 2B. Where a two-piece device is provided for surgical method 300, some steps illustrated in FIG. 3A may be described in one or more additional steps than those of a one-piece device. Additionally, the particular device used in method 300 can differ based on the size and needs of the particular patient. Both the size of the device's hollow member and non-expandable portion/component may need to be modified to provide the patient with the best chance of success. Additionally, if a particular weight loss goal is desired, a certain amount of abdominal pressure may be needed from the device. In those cases, a larger hollow member, a stronger non-expandable portion/component, and/or a different or stronger coupling or attachment means may be necessary or desired to meet weight loss and intra-abdominal pressure needs.

Returning to FIG. 3A, at 309 the expandable implantable food restriction device is inserted into the body via the incision created at 303. The expandable food restriction device of the present disclosure may be inserted via any suitable method for same, including, without limitation, via endoscope or open method technique. At 312, the expandable implantable food restriction device is positioned to a point superficial to the abdominal cavity. The particular point superficial to the abdominal cavity may be chosen with specific regard to the anatomy of the patient on which method 300 is being performed. Additionally, in instances where the expandable food restriction device comprises two pieces (i.e., a hollow member and mesh), the hollow member can be positioned at the appropriate location without initial regard to the separate, non-expandable component of the device.

At 315, the expandable implantable food restriction device is attached to body at the particular position superficial to the abdominal cavity. For the one-piece embodiment of the expandable device, the relatively non-expandable portion of the hollow member can be placed against the fascia and coupled thereto via the coupling or attachment means of the device. In the one-piece device, the periphery or edges of the non-expandable portion can be placed against the fascia, and the coupling or attachment means can be used to securely attach or fasten the device to fascia, ensuring that the hollow member is not moved or displaced from the location the device was positioned at 312 during the process of expanding or contracting the hollow member, or during any other normal and/or predictable movements of the body or abdominal cavity. When a two-piece embodiment of the expandable device is provided for method 300, the relatively non-expandable component of the device is placed on top of the properly positioned hollow member (positioned at 315), and the relatively non-expandable component is attached to the fascia such that the hollow member can remain in the proper selected position during normal operations of the device (i.e., expansion and contraction) and after normal and/or predictable movements of the abdominal region. Using either embodiment, the expandable device should be securely fastened to the position superficial to the fascia (or another more appropriate position, if available) to ensure that a pocket is formed for the hollow member such that (1) the hollow member remains relatively stationary at the selected position, and (2) the hollow member is capable of expanding while being relatively restricted from expanding in the direction of the relatively non-expandable portion/component so that the intra-abdominal pressure created during expansion (and remaining after said expansion) is directed towards the abdominal cavity which in turn applies pressure to the peritoneum. Once the expandable implantable food restriction device is attached to the point superficial to the abdominal cavity, at 318 the incision (created at 303) is closed.

Once the food restriction device has been inserted into the body and attached to the point superficial to the abdominal cavity, results attributed to the restriction device are evaluated after a medically relevant period of time, as illustrated at 321. The medically relevant period of time may be determined on a case-by-case basis according to the patient, the invasiveness of the procedure used to insert the device, the results expected from the addition of the device, as well as any other factors, including the normal follow-up time for procedures of this type. In some instances, the device may be inserted, and other than normal check-ups, no further evaluations may be necessary, and evaluations may occur in intervals, or when otherwise necessary. In cases where substantial weight loss is expected and/or higher amounts of intra-abdominal pressure is generated or desired, evaluations may occur at more closely-spaced intervals in order to carefully monitor the weight loss associated with the device.

After the evaluations, it is determined whether the intra-abdominal pressure provided by the device is sufficient or needs adjustment at 324. Sufficiency may be based on a patient's weight loss (or lack thereof) attributed to the device, particularly according to a pre-determined weight loss schedule, the desire for more or less weight loss in the patient, the effects of the device, or any other metric by which the success of the device is measured. If it is determined that the intra-abdominal pressure is sufficient, the results will be evaluated again after another medically relevant period of time. The time between the initial evaluation and later evaluations may vary on a case-by-case basis. For example, a first evaluation may occur shortly after the device is inserted, while a second (or later) evaluation may occur after longer intervals. If, however, it is determined that a change in intra-abdominal pressure is necessary, method 300 continues at 327.

At 327, the hollow member of the expandable version of the implantable food restriction device is selectively expanded or contracted via the means for expansion and contraction to exert additional or less pressure on the abdominal cavity according to the determination from steps 321 and 324. In some embodiments, the device may include a magnetic injection port, such that a magnet can locate the position of the port. Once located, a syringe, needle, or other similar device can be used to add or remove the appropriate fluid or gas to or from the hollow member to increase or decrease, respectively, the intra-abdominal pressure exerted by the device. In another embodiment, a relatively hollow and flexible tube protruding from, or contained within, the body and attached to the hollow member of the restriction device can be accessed to add or remove the fluid/gas used with the device. After the intra-abdominal pressure is changed at 327, the effects of the device are reevaluated at step 321 at some medically relevant time after the change is made. The time between any expansion or contraction of the device and further evaluation may be determined on a case-by-case basis, and again, can be determined based on a medically relevant period, as well as based on the patient's personal weight loss timetable.

A particular advantage of the expandable device is that once a particular weight loss amount or number is reached, and further modifications to intra-abdominal pressure is no longer desired or necessary, a second operation may be performed (not illustrated) to remove the hollow member of the device. In that surgical method, an incision to the body is made and the hollow member of the device is emptied. In some instances, the hollow member of the device may be emptied prior to the incision. An incision to the relatively non-expandable portion/component of the device can be made, and the deflated or empty hollow member may then be removed from the device. In the one-part embodiment of the device, the hollow member may be cut or detached from the relatively non-expandable portion, while in the two-piece embodiment, the hollow member may be removed through the incision in the relatively non-expandable component. In both embodiments of the device, the relatively non-expandable portion/component may remain attached to the fascia during the second procedure. Thus, once the hollow member is removed, the incision to the relatively non-expandable portion/component of the device can be closed (e.g., by placating the relatively non-expandable portion/component and closing the incision via suture, surgical staple, or the like), or a second relatively non-expandable component may be added or overlaid to the device to cover the incision into the first relatively non-expandable component. The second relatively non-expandable component may be similar to the passive restriction device described in FIG. 2C, such as surgical mesh or the like. In either embodiment, the device (minus the hollow member) can be kept in the abdominal wall to maintain the intra-abdominal pressure generated by the device prior to its removal. In some instances, the relatively non-expandable portion/component may need to be tightened or reattached via the coupling or attachment means of the device in order to replicate the pressure achieved prior to the removal and/or deflation of the hollow member. This secondary method allows the effects of the device to be retained, even after the hollow member of the device has been removed. As such, the effects and benefits of the previously expandable device may be maintained via the now-passive food restriction device retained within the body for potentially long periods of time.

FIG. 3B illustrates one embodiment of a surgical method 335 for controlling weight of a body using one or more passive versions of the disclosed device. Upon placement, a passive version of the restriction device exerts intra-abdominal pressure, reducing the volume of the abdominal cavity and thus reducing the amount of food required to produce satiety. Surgical method 335 illustrates the use of supplementary passive restriction devices over a period of time (in lieu of the expansion or contraction of the expandable version of the device) to increase the intra-abdominal pressure exerted on the abdominal cavity in order to achieve the weight loss and other medical goals of the device's use.

Turning to FIG. 3B at 338, a first incision into the abdominal region of a body is made. As in 303 of FIG. 3A, the incision can be made utilizing any traditional means for same, including via scalpel, laser, or other suitable cutting device. At 341, a first passive implantable food restriction device is provided. The first passive implantable food restriction device may be the device described in FIG. 2C (i.e., a relatively non-expandable component and coupling or attachment means), or it may be similar to the expandable device of FIGS. 2A and 2B (i.e., including a hollow member, a relatively non-expandable portion/component, and coupling or attachment means, where the hollow member is not expanded or contracted within the body). The particular first passive implantable food restriction device can differ based on the size and needs of the particular body (e.g., according to the necessary elasticity required, the specific issues the device is meant to correct, and/or the desired weight loss goals of the patient, as well as other relevant criteria).

At 344, the first passive implantable food restriction device is inserted into the body via the incision created at 338. The first passive food restriction device may be inserted via any suitable method for same, including, without limitation, via endoscope or open method technique. At 347, the first passive restriction device is positioned to or around a point superficial to the abdominal cavity. Similar to the expandable device, the particular point may be selected on a case-by-case basis, with specific regard to the body of the patient on which method 335 is being performed. In some instances, the position selected may be the position that will allow the greatest amount of intra-abdominal pressure to be exerted, while in other instances, the position selected may be a position that is capable of allowing the device to generate additional intra-abdominal pressure, while still remaining relatively comfortable and/or easy to access for future procedures and/or monitoring.

At 350, the first passive implantable food restriction device is attached to the body at (or around) the selected position superficial to the abdominal cavity. For passive devices comprising only the relatively non-expandable component and the coupling or attachment means, the passive device can be generally centered or located around the selected position, and circumferentially attached to the abdominal wall (i.e., the fascia). In passive device embodiments including a hollow member, the hollow member may be centered on the selected position, and the relatively non-expandable portion of the device may be placed against the fascia and coupled or attached thereto via the coupling or attachment means. In any embodiment, the passive restriction device should be securely fastened to the body to ensure that the device remains relatively stationary during the normal course of movement and physical actions associated with the selected location and surrounding area. In some instances, the passive device may be attached to the body in a manner similar to that described at 315 of FIG. 3A. Once attached, the passive device alone increases the intra-abdominal pressure within the body and is directed inwardly (in view of the body) towards the abdominal cavity. Once the passive implantable food restriction device is attached to the point superficial to the abdominal cavity, at 353 the incision (created at 338) is closed.

Once the passive implantable food restriction device has been inserted into the body and attached to the point superficial to the abdominal cavity, results attributed to the restriction device are evaluated after a medically relevant period of time, as shown at 356. The medically relevant time period may, as with the expandable device, be determined on a case-by-case basis according to the patient, the invasiveness of the procedure used to insert the device, the results expected from the addition of the device, as well as any other factors, including the normal follow-up time for similar procedures. In some instances, the device may be inserted, and, other than normal check-ups, no further evaluations may be necessary. The device and its effects may be evaluated during the patient's check-up, or if other issues occur. In cases where substantial weight loss is expected, higher amounts of intra-abdominal pressure are generated or desired, or the patient's condition or background indicates the need for additional or more frequent review, evaluations may occur at more closely-spaced intervals.

After the evaluations, it is determined whether the intra-abdominal pressure provided by the device is sufficient or needs adjustment at 359. Sufficiency may be based on a patient's weight loss (or lack thereof) attributed to the device, as well as other relevant metrics (such as those described at 321 and 324 of FIG. 3A). If it is determined that the intra-abdominal pressure is sufficient, the results will be evaluated again after another medically relevant period of time (which may differ between each evaluation based on any number of factors), and method 335 returns to 356. If, however, it is determined that a change in intra-abdominal pressure is necessary, method 335 continues at 362.

The portion of method 335 illustrated in steps 362-377 describes the cascading addition of one or more passive implantable food restriction devices into the body in order to gradually and periodically increase the intra-abdominal pressure of the patient. In some cases, the second (or successive) passive device may be added at a much later time than when the earlier passive device(s) was (were) inserted. This may be the case in instances where a patient's weight loss goal is originally met by the initial device, but said goal is later revised based on various medical or non-medical factors. Additionally, while described as inserting additional passive devices, it will be understood that in some cases one or more of the passive devices may need to be removed from the body. For instance, if a female patient intends to or does become pregnant, the implantable device (whether passive or expandable) may need to be removed or released (e.g., by detaching one or more of the attachment means from the body in order to decrease the total pressure generated by the device to an appropriate level) in order to allow the natural expansion of the abdominal cavity associated with pregnancy and the growth of the fetus. Additionally, if a patient outgrows the original (or successive) device(s) even after the increased intra-abdominal pressure, the original (and/or one or more of the successive) device(s) may need to be removed in order to avoid complications from the device. In other similar or analogous cases, the device may not need to be removed. Still further, it is contemplated that steps 362-377 of FIG. 3B may be used to supplement a previously-inserted expandable implantable food restriction device. For instance, the new passive device may tighten or make smaller the pocket created for the hollow member of the expandable device, so that any expansions to the hollow member cause a greater increase in intra-abdominal pressure than if the passive device had not been added. Therefore, in some embodiments, the steps discussed at 362-377 may be used with the surgical method described in FIG. 3A. Still further, any and all permutations and combinations of passive and expandable devices can be used as appropriate to meet the medical, physiological, and aesthetic needs and goals of both patient and physician/surgeon. For example, an expandable implantable food restriction device may be added at 362-377 after one or more passive devices have been inserted to the body, and in other instances, after both an expandable and passive device have been inserted.

Returning to FIG. 3B, at 362 a new incision is made to the abdominal region of the body. In some instances, the new incision may be made at the same location as a previous incision to add the original (or a successive) passive restriction device. This may be aesthetically optimal in order to reduce the scars or other indicia of said incisions on the body. At 365, a new passive implantable food restriction device is provided. Generally, the device provided at 365 will comprise a non-expandable component and coupling or attachment means similar to the device described in FIG. 2C. Further, the new passive device (or at least the relatively non-expandable component of said device) may need to be larger than the previously applied passive device in order for the new passive device to be attached properly to a point superficial to the abdominal cavity (such as the fascia).

At 368, the new passive implantable food restriction device is inserted into the body via the incision created at 362. The new passive food restriction device may be inserted via any suitable method for same, including, without limitation, via endoscope or open method technique, and may, in some cases, be a different method than that used in inserting the original (or any previous) passive device. At 371, the new passive restriction device is positioned around a point superficial to the abdominal cavity, and in most cases, around the originally (and any successive) inserted passive device. By adding the device around the previously inserted passive devices, the intra-abdominal pressure can be increased, and the new device easily attached superficial to the abdominal cavity, and specifically, the fascia. In some instances, it may be advantageous to position the new passive restriction device to a point superficial to the abdominal cavity that does not directly overlay the original (or any previous) passive device. In those cases, the new passive restriction device will be located where deemed appropriate for optimum success in light of all factors associated with the procedure.

Once positioned appropriately, the new passive implantable food restriction device is attached to the body at (or around) the newly selected position superficial to the abdominal cavity at 374. As described in 350, the passive device can be attached to the selected point via the coupling or attachment means of the device, such that the device is securely fastened to the body. For instance, the new passive device may be circumferentially attached to the fascia and may surround the original (or previous) passive device. In another instance, some portions of the new device may overlap the original (or previous) passive device, and attachment to the body may include a portion of the new device being attached directly to the body while other portions are attached or coupled to the previous device. Other methods and arrangements for attachment may also be used as appropriate. Once attached, the new passive device increases the intra-abdominal pressure within the body that is directed inward towards the peritoneum and, ultimately, the abdominal cavity. Once the passive implantable food restriction device is attached to the body, the new incision (created at 362) is closed at 377.

After addition of the new passive implantable food restriction device, method 335 returns to 356 where the results attributed to the combination of the original and new restriction device are evaluated after a medically relevant period of time. Upon evaluation, it is determined whether the intra-abdominal pressure provided by the device is sufficient or needs adjustment at 359. If the intra-abdominal pressure is determined to be sufficient, the results will be evaluated again after another medically relevant period of time (which may differ between each evaluation based on any number of factors), returning method 335 to 356. If, however, it is determined that a change in intra-abdominal pressure is necessary, method 335 continues at 362, and another passive device may be added using steps 362-377.

FIG. 4A is an illustration of layers of the abdominal region 20 of a body 10 after placement and attachment of an embodiment of the one-piece expandable version of the food restriction device 90 (of FIG. 2A) in the device's 90 expanded mode. As the hollow member 92 of the food restriction device 90 is expanded via the means for expansion and contraction (not shown), in a preferred embodiment the relatively expandable portion 110 of the hollow member 92 expands and directs the pressure created by compliance of the non-expandable portion of the food restriction device 90 towards the peritoneum 70 and, ultimately, the abdominal cavity 80, the overall effect of which is to reduce the relative volume of the abdominal cavity 80 without the need for physical invasion of the abdominal cavity 80. Specifically, and as illustrated, the arc length of the fascia 60 is decreased in the body 10, such that the fascia 60 is relatively linear in direct contrast to its previous shape as shown in FIG. 2A. The expansion thus provides the patient with an abdominal wall similar to that of a normal sized body as illustrated in FIG. 1A. With the relative volume of the abdominal cavity 80 reduced, the body 10 will require less food to achieve a sense of satiety. Additionally, the ingestion of less food by the body 10 will also necessarily advantageously affect relative food absorption. The combined effect of the foregoing will be a reduction in weight of the body 10.

In use, the expandable food restriction device 90 is positioned to a point superficial to the abdominal cavity 80. Specifically, in a preferred embodiment, the expandable food restriction device 90 is positioned to a point superficial to the fascia 60 of the abdominal region 20. This positioning of the food restriction device 90 is considered an optimal balance of the desired transfer of pressure to the abdominal cavity 80 with the desire to have the least physical invasion of the abdominal region 20. Although optimal positioning of the food restriction device 90 is described as being superficial to the fascia 60 of the abdominal region 20, it is noted that the present disclosure contemplates positioning of the food restriction device 90 at any point within the abdominal region 20 that reduces the relative volume of the abdominal cavity 80 via the production of intra-abdominal pressure without physical invasion of the abdominal cavity 80, including, without limitation, points deep to the skin 30 and the fat 40 of the abdominal region 20, but (a) superficial to the fascia 60, the muscle 50 and the peritoneum 70 of the abdominal region 20; or (b) underneath the fascia 60; or (c) within the peritoneal cavity.

As can be appreciated, the change in relative volume of the abdominal cavity 80 created as a result of the food restriction device 90 of the present disclosure is directly related to the relative volume of the food restriction device 90 employed. Therefore, if a passive version of the food restriction device 90 (such as those described in relation to FIG. 2C) is utilized, the relative shape and size of the device (such as the relatively non-expandable component 100) can be varied to effectuate the desired change in volume of the abdominal cavity 80. If an expandable version of the food restriction device 90 is utilized, again the relative shape, construction (e.g., relative position of the relatively expandable portion 110 and/or the relatively non-expandable portion 100), and degree of expansion can be varied to effectuate the desired change in volume of the abdominal cavity 80 and relative arc length of the abdominal wall and fascia 60. For example, with respect to the expandable version of the food restriction device 90, if additional weight loss by the body 10 is desired, the relative volume of the hollow member 92 of the food restriction device 90 can be increased via the means for expansion and contraction, such that the arc length of the fascia 60 is made additionally linear. Conversely, if it is desired that the body 10 experience less weight loss (or have the rate of weight reduction slowed), the relative volume of the hollow member 92 of the food restriction device 90 can be reduced via the means for contraction and expansion in order to allow the fascia 60 to expand its relative arc length.

FIG. 4B illustrates the effect of the expanded mode of the two-piece restriction device 90 illustrated in FIG. 2B. The expansion of its hollow member 92 also performs the same function as the expanded one-piece device 92 of FIG. 4A—the relative shortening of the abdominal wall or fascia 60 due to the expansion of the hollow member 92. The fascia 60 as illustrated is represented in a relatively linear fashion as compared to the previously obese body 10 of FIG. 2B, indicating that the shortening of the fascia 60 and the relative reduction of volume in the abdominal cavity 80 assists in weight-loss and reinforcement or decreased compliance of the abdominal wall.

FIG. 4C is an illustration of layers the abdominal region 20 of a body 10 including placement and attachment of two cascading or overlapping versions of the passive implantable food restriction devices 90 (illustrated in FIG. 2C) and as inserted according to FIG. 3B. The first and originally inserted passive device 90A comprises a first relatively non-expandable component 100A and first coupling or attachment means 94A, and the second, later-added passive device comprises a second relatively non-expandable component 100B and a second coupling or attachment means 94B. Further, the second passive device 90B is illustrated with its relatively non-expandable component 100B being larger in length than the relatively non-expandable component 100A of the first device 90A. In some cases, the second passive device 90B may be the same size or smaller than the first device 90A as appropriate.

As illustrated, the combination of two (or more) passive implantable food restriction devices 90A and 90B increases the intra-abdominal pressure in the abdominal region 20 by shortening the arc length of the abdominal wall, and directs said pressure towards the peritoneum 70 and, ultimately, the abdominal cavity 80, the overall effect of which is to reduce the relative volume of the abdominal cavity 80. While only two passive devices are illustrated in FIG. 4B, three or more devices may be added as needed (using, for example, the method described in FIG. 3B). In some instances, but not shown here, the relatively non-expandable component 100B of the second passive device 90B may be in (at least partial) contact with the relatively non-expandable component 100A of the first passive device 90A, such that the pressure exerted by the second device 90B is exerted directly upon the first device 90A. Additionally, in some instances the second passive device 90B may be oriented apart from, or not overlaid on, the first passive device 90A. In those instances, the intra-abdominal pressure exerted by the plurality of devices may cause the abdominal cavity 80 to be compressed in a different manner or shape than that illustrated in FIG. 4B. In either instance, the arc length of the fascia 60 is shortened, thus producing a relatively linear orientation of the abdominal wall and therefore, increased pressure towards the abdominal cavity 80.

One significant advantage of the devices and methods of the present disclosure is that the abdominal cavity 80 is not invaded. The lack of physical invasion of the abdominal cavity 80 eliminates leaking of stomach juices into the abdomen, injury to internal organs (e.g., spleen) and further avoids complications associated with the use of devices within the abdominal cavity 80 such as staples, bands and the like, thus leading to quicker recovery times, reduced hospital stays, and increased patient satisfaction.

A number of embodiments have been described in the present disclosure. Nevertheless, it will be understood that various modifications may be made without departing from the spirit and scope of the described devices and methods. For example, the device could be shaped or otherwise modified (e.g., in size) so as to more completely focus the pressure created via expansion of same. Further, while the illustrated embodiments generally represent the methods and device used in an obese patient, this disclosure also contemplates the use of the device in non-obese individuals. For example, mothers after childbirth may be candidates for use of the device and method in order to reverse or correct the effects of pregnancy on the abdominal wall (e.g., increased compliance due to stretching, tearing, or failure of the abdominal wall). Additionally, non-obese, but otherwise over-weight individuals may also benefit from the effects and purpose of the device. Further, any other suitable patients may candidates for the illustrated devices and methods on a case-by-case basis. Accordingly, other embodiments are within the scope of the following claims.

What is claimed is:

1. An implantable device for use with a surgical method for controlling weight of a body, said implantable device comprising:
a hollow member;
said hollow member including an exterior surface having an expandable portion;
said exterior surface further including a non-expandable portion;
means for expansion and contraction of the hollow member; and
attachment means for coupling the non-expandable portion of the exterior surface to a portion of the body superficial to the abdominal cavity such that pressure exerted by the expandable portion, when the expandable portion is expanded, is directed to the abdominal cavity.

2. The implantable device of claim 1, wherein the expandable portion and the non-expandable portion cover unequal portions of the exterior surface of the hollow member.

3. The implantable device of claim 2, wherein the non-expandable portion is partially attached to one side of the hollow member and includes a portion separate from the exterior surface of the hollow member.

4. The implantable device of claim 3, wherein the attachment means for coupling the non-expandable portion of the exterior surface of the device to the portion of the body superficial to the abdominal cavity are integrally formed with the non-expandable portion of the exterior surface.

5. The implantable device of claim 1, wherein the means for expansion and contraction includes means for introduction of a liquid or gas to the hollow member to expand the hollow member.

6. The implantable device of claim 5, wherein the introduction of a fluid or gas is accomplished via a hollow tube or a magnetic injection port.

7. The implantable device of claim 1, wherein the means for expansion and contraction includes means for extraction of a liquid or a gas from the hollow member to contract the hollow member.

8. The implantable device of claim 7, wherein the extraction of a fluid or a gas is accomplished via a hollow tube or a magnetic injection port.

9. The implantable device of claim 1, wherein the expandable portion and the non-expandable portion are shaped to direct location of pressure created from expansion of the hollow member.

10. The implantable device of claim 1, wherein the portion of the body superficial to the abdominal cavity comprises the fascia of the body.

11. The implantable device of claim 1, wherein the attachment means for coupling the device to the portion of the body superficial to the abdominal cavity are operable to couple the periphery of the non-expandable portion of the hollow member to the portion of the body superficial to the abdominal cavity.

12. The implantable device of claim 11, wherein the attachment means for coupling the device to the portion of the body superficial to the abdominal cavity are continuously located along the periphery of the non-expandable portion of the hollow member.

13. The implantable device of claim 11, wherein the attachment means for coupling the device to the portion of the body superficial to the abdominal cavity are periodically located along the periphery of the non-expandable portion of the exterior surface of the hollow member.

14. The implantable device of claim 1, wherein the non-expandable portion of the exterior surface of the hollow member comprises surgical mesh.

15. The implantable device of claim 1, wherein the attachment means for coupling the device to the portion of the body superficial to the abdominal cavity comprises medical sutures.

16. An implantable device for use with a surgical method for controlling weight of a body, said implantable device comprising:
a hollow member comprising:
an expandable exterior surface; and
means for expansion and contraction of the hollow member;
wherein the hollow member is configured to be placed at a point superficial to the abdominal cavity of the body;
a non-expandable component for constricting the hollow member to the point superficial to the abdominal cavity of the body, the non-expandable component placed at a location opposite the hollow member with respect to the abdominal cavity of the body; and
attachment means for coupling the non-expandable component to a portion of the body superficial to the abdominal cavity, wherein the attachment means for coupling fasten the non-expandable component to a location relatively circumferential to the hollow member.

17. The implantable device of claim 16, wherein the attachment means for coupling the non-expandable component to the portion of the body superficial to the abdominal cavity comprises a part of the non-expandable component.

18. The implantable device of claim 16, wherein non-expandable component comprises relatively non-expandable surgical mesh.

19. The implantable device of claim 16, wherein the attachment means comprises medical sutures.

20. The implantable device of claim 16, wherein the non-expandable component and attachment means form a pocket between the non-expandable component and the point superficial to the abdominal cavity, wherein the pocket restricts the movement of the hollow member to the point superficial to the abdominal cavity of the body when the hollow member is expanded via the means for expansion and contraction, and such that pressure exerted by the expandable hollow member, when the expandable hollow member is expanded, is directed to the abdominal cavity and away from the non-expandable component.

21. The implantable device of claim 16, wherein the means for expansion and contraction includes means for introduction of a liquid or gas to the hollow member to expand the hollow member.

22. A surgical method for controlling weight of a body, said body including an abdominal region having skin, fat, muscle, fascia, a peritoneum and an abdominal cavity, the method comprising the steps of:
making a first incision into the abdominal region of the body;
providing a first implantable food restriction device;
inserting the first implantable food restriction device into said incision;
positioning the first implantable food restriction device to a point superficial to the abdominal cavity to reduce the compliance of the abdominal wall and direct pressure to the abdominal cavity;
attaching the first implantable food restriction device to the point superficial to the abdominal cavity;
closing the incision into the abdominal region of the body;
evaluating the effect of the first implantable food restriction device after a medically relevant period of time to determine whether additional pressure is to be exerted and directed to the abdominal cavity; and
if additional pressure is to be exerted and directed to the abdominal cavity,
making a second incision into the abdominal region of the body;
providing a second implantable food restriction device;
inserting the second implantable food restriction device into said incision;
positioning the second implantable food restriction device to a point superficial to the abdominal cavity to exert and direct pressure to the abdominal cavity and overlaying the first implantable food restriction device;
attaching the second implantable food restriction device to the point superficial to the abdominal cavity; and
closing the second incision into the abdominal region of the body.

23. The surgical method of claim 22, wherein:
the first implantable food restriction device comprises:
a hollow member;
said hollow member including an exterior surface having an expandable portion;
said exterior surface further including a non-expandable portion; and
first attachment means for coupling the device to a portion of the body superficial to the abdominal cavity such that pressure exerted by the expandable portion, when the expandable portion is expanded, is directed to the abdominal cavity; and
the second implantable food restriction device comprises a non-expandable component and second attachment means for coupling the second implantable food restriction device to a portion of the body superficial to the abdominal cavity.

24. The surgical method of claim 22, wherein the step of positioning the second implantable food restriction device to a point superficial to the abdominal cavity to exert and direct pressure to the abdominal cavity and overlaying the first implantable food restriction device comprises positioning the second implantable food restriction device to a point directly on top of, but expanding past, the first implantable food restriction device.

25. The surgical method of claim 22, wherein the step of positioning the first implantable food restriction device includes locating the implantable food restriction device at a point deep to the skin and the fat of the abdominal region and the peritoneum of the abdominal region.

26. The surgical method of claim 22, wherein:
the step of positioning the first implantable food restriction device further includes orienting the first implantable food restriction device such that pressure created by first implantable food restriction device is directed towards the abdominal cavity; and
wherein the step of positioning the second implantable food restriction device further includes orienting the second implantable food restriction device such that pressure created by the second implantable food restriction device is directed towards the abdominal cavity and the first implantable food restriction device.

27. The surgical method of claim 22, wherein endoscope or open technique is utilized to implant the first food restriction device.

28. The surgical method of claim 27, wherein endoscope or open technique is utilized to implant the second food restriction device.
